(19) United States
(12) Patent Application Publication
(10) Pub. No.: US 2015/0157196 A1
(43) Pub. Date: Jun. 11, 2015

(54) FLUSHED MEDICAL CONNECTOR WITH OPTICAL AND ELECTRICAL CONNECTIONS

(71) Applicant: KONINKLIJKE PHILIPS N.V., Eindhoven (NL)

(72) Inventors: Martinus Bernardus Van Der Mark, Best (NL); Gert Wim 'T Hooft, Eindhoven (NL); Heinrich Johannes Eckhard Von Busch, Erlangen (DE)

(21) Appl. No.: 14/405,267

(22) PCT Filed: Jun. 5, 2013
(86) PCT No.: PCT/IB2013/054632

§ 371 Date: Dec. 3, 2014

Related U.S. Application Data
(60) Provisional application No. 61/656,544, filed on Jun. 7, 2012.

(51) Int. Cl. A61B 1/12; H01R 43/00; A61B 1/00

(52) U.S. Cl. CPC A61B 1/126; A61B 1/00114; A61B 1/00124; H01R 43/002

(57) ABSTRACT

A medical connector receptacle includes a receptacle (134) configured to receive a proximal end portion (106) of a medical device. An inlet port (102) in fluid communication with the receptacle is configured to receive a fluid for cleaning the proximal end portion. An outlet port (124) in fluid communication with the receptacle is configured to eject the fluid and debris from the receptacle. An active connection point (128) is configured to mate with the proximal end portion of the medical device such that the fluid removes the debris to permit an improved connection.

This disclosure relates to medical instruments and more particularly to connector devices and receptacles configured with fluid passages for flushing connection points.

Many advantages exist for replacing conventional surgical procedures with minimally invasive (MI) interventions including, e.g., reduced trauma, reduced duration of a hospital stay and reduced cost. To enable further innovation in medical instrumentation—thus enabling more advanced and more challenging MI interventions—there is a need to integrate miniature sensors for in-body imaging and physiological measurement in instruments like catheters and guide wires.

Electrical and optical signal delivery to and from long and thin devices such as a medical catheter or guide wire for imaging, sensing, sensitizing or even ablation is becoming more common. Guide wires are used in medical interventions to position devices, including sheaths and catheters in a patient during a procedure. It is often necessary to slide devices over a proximal end of a guide wire into and out of the patient. During retraction of a device over the guide wire, the proximal end of the guide wire will become contaminated, for example, with blood. When such a guide wire includes active elements, there may be electrical and/or optical contacts at the proximal end of the guide wire. These connections need to make proper contact to a contra connector (e.g., a receiving end), which connects to a driving or sensing instrument of the active elements in the guide wire. These contacts may also become contaminated. Instruments may need to glide over the guide wire into and out of the body. The sterility of the guide wire, in particular its proximal end, needs to be guaranteed at all times. This is challenging if the proximal end also needs to be connected to and disconnected from an external optical or electronic device.

In accordance with the present principles, a medical connector receptacle includes a receptacle configured to receive a proximal end portion of a medical device. An inlet port in fluid communication with the receptacle is configured to receive a fluid for cleaning the proximal end portion. An outlet port in fluid communication with the receptacle is configured to eject the fluid and so remove debris from the receptacle. A connection point is configured to mate with the proximal end portion of the medical device such that the fluid removes the debris to permit an improved connection.

A system for cleaning connections includes a receptacle configured to receive a proximal end portion of a medical device. The receptacle includes at least one inlet port, at least one outlet port and at least one active connection point configured to mate with the proximal end portion of the medical device. At least one valve is coupled to the at least one inlet port, and the at least one valve is controlled to enable or disable a fluid jet to clean the proximal end portion. A sensor is disposed within the receptacle to enable the at least one valve when the proximal end portion is sensed in the receptacle.

A method for preparing a connector for connection includes inserting a connecting end portion of a connection device in a receptacle; advancing the connecting end portion into the receptacle to a first position corresponding with a first inlet port; streaming a first fluid jet into the first inlet port to douse the connecting end portion; and clearing the receptacle to improve contact between active connections of the connecting end portion and active connections of the receptacle.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

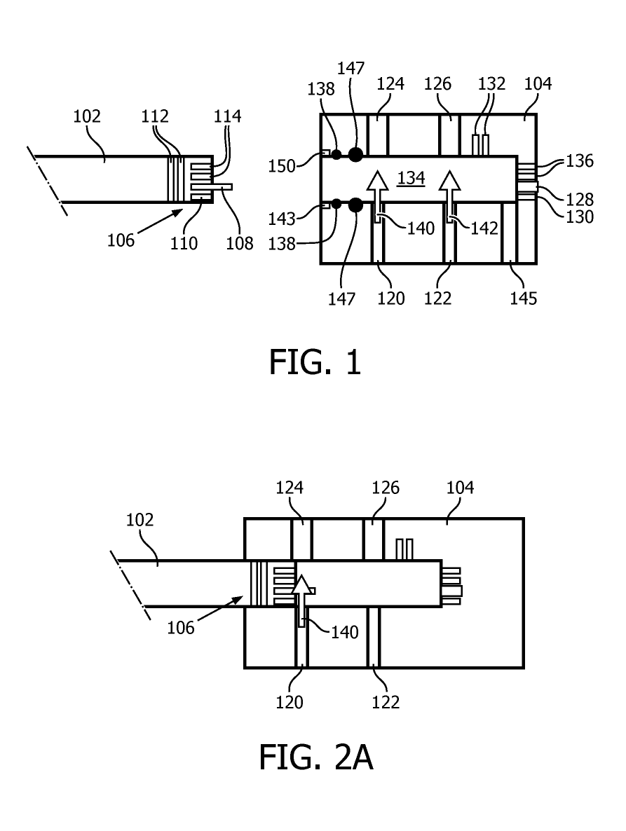

FIG. 1 is a cross-sectional view of a connection end portion of a medical device and a mating receptacle of a contra connection device in accordance with one illustrative embodiment;

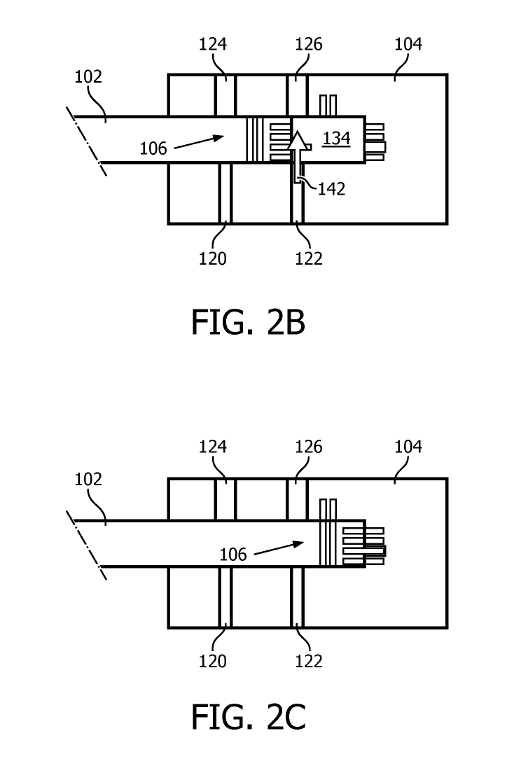

FIG. 2A is a cross-sectional view of the connection end portion of the medical device of FIG. 1 advanced to a first inlet position in the mating receptacle of the contra connection device for flushing with a first jet in accordance with one illustrative embodiment;

FIG. 2B is a cross-sectional view of the connection end portion of the medical device of FIG. 2A advanced to a second inlet position in the mating receptacle of the contra connection device for flushing or drying with a second jet in accordance with one illustrative embodiment;

FIG. 2C is a cross-sectional view of the connection end portion of the medical device of FIG. 2B advanced so that active connection points of the connection end portion and the receptacle are contacted in accordance with one illustrative embodiment;

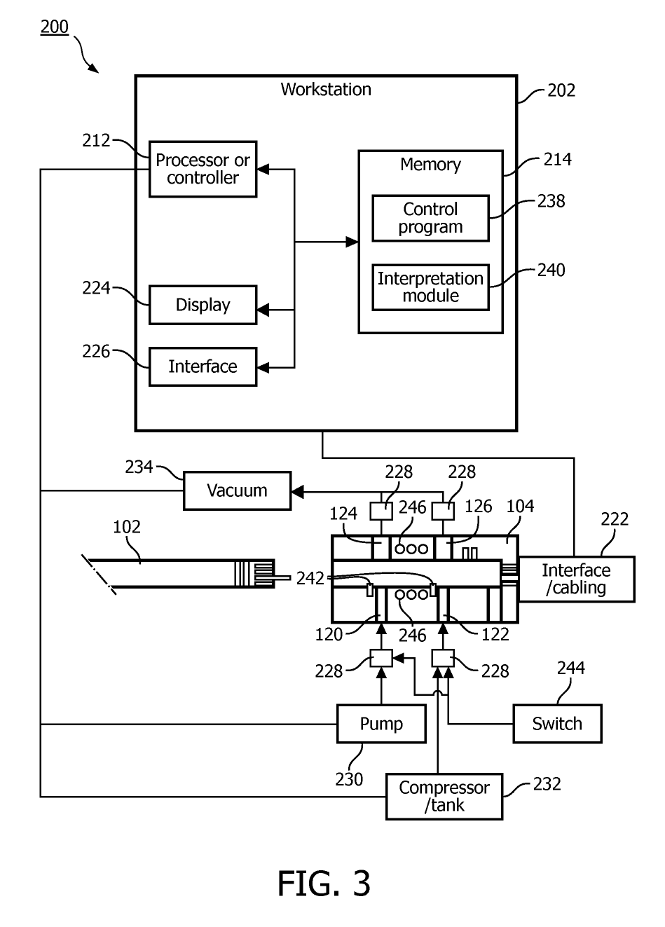

FIG. 3 is a block/flow diagram showing a system for preparing a connector for connection with a receptacle in accordance with one embodiment; and

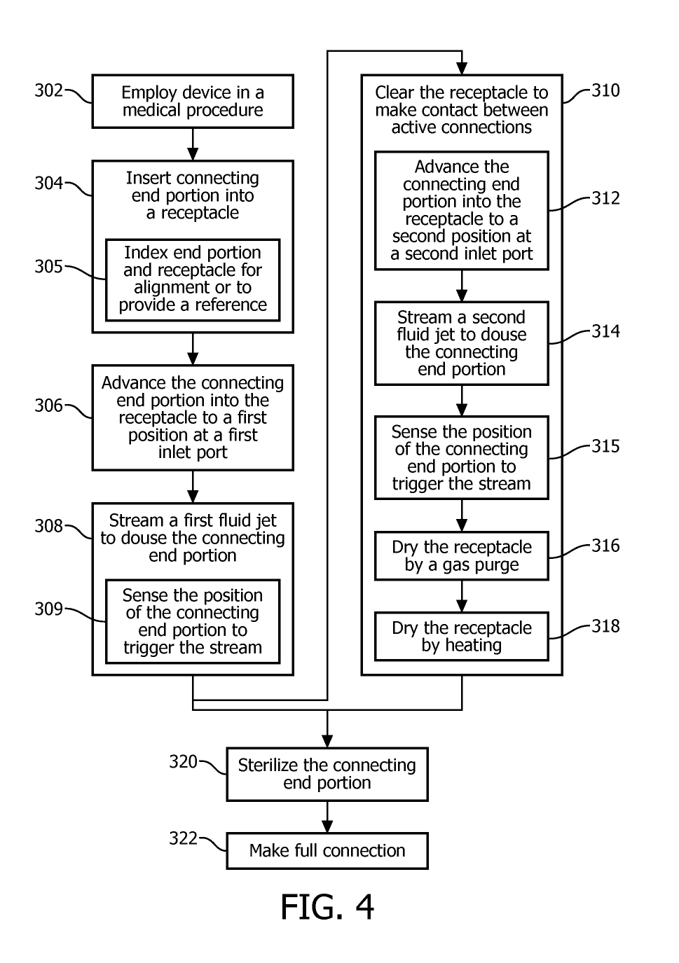

FIG. 4 is a flow diagram showing a method for preparing a connector for connection with a receptacle in accordance with illustrative embodiments.

In accordance with the present principles, active connections for interventional devices are provided with a cleaning or flushing mechanism to ensure that a proximal end portion of an interventional device and, in particular, an active connection portion is cleaned and/or sterilized. In one example, a guide wire is employed in medical interventions. The guide wire may include a connector portion with active elements, e.g., one or more electrical or optical contacts at its proximal end. The connector portion with one or more active elements may be received in a contra connector where corresponding active elements are connected with the active elements of the connector portion of the guide wire. The contra connector includes orifices or ports for receiving and expelling fluids. The fluids are employed in a single step or in multiple steps to clean and/or sterilize a proximal end portion of the guide wire or other devices. The fluid may include, for example, water, air, a non-conductive and non-corrosive cleaning agent in the contra connector. The fluid is ejected from the contra connector after the insertion of the proximal end of the guide wire to guarantee a proper connection.

It should be understood that the present invention will be described in terms of medical instruments; however, the teachings of the present invention are much broader and are applicable to any instruments with active connectors that would benefit from being cleaned prior to connection. In some embodiments, the present principles are employed in interventional procedure devices or devices for analyzing complex biological or mechanical systems. In particular, the present principles are applicable to internal devices for performing surgery on biological systems in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

Referring now to the drawings in which like numerals represent the same or similar elements and initially to FIG. 1, a connection system 100 is illustratively shown in accordance with one embodiment. A device 102, such as, a medical device may include a guide wire, catheter, endoscope, probe, robot, electrode, filter device, balloon device, or other medical component, etc. The device 102 has a distal end portion (not shown) that is inserted into a patient or system to be analyzed, repaired or treated. The device 102 includes a proximal end portion 106 configured with one or more active contacts. The active contacts may include optical fiber or light guide connections 110, electrical connections 114 (e.g., power and/or signal), electrical pin connections 108, lateral electrical connections 112, electrical pads, mechanical elements 143, such as, locks, torque stops, fluid connections 120, 122, etc. (such as for irrigation or cooling of the distal tip of the device), a central or side lumen or port 145 (for introducing other devices, even when the connection end portion of the medical device is in the receptacle), etc. Other structures are contemplated and may be employed in accordance with the present principles. For example, a feed, clamp and eject mechanism may be employed using gas pressure regulation through e.g., port 145 inside the receptacle 104 may be employed. A sensor 150 in the input port of the receptacle is placed to respond to the initial insertion or retraction of the medical device by an operator. In another embodiment, an automatic feed, clamp and eject mechanism may include motorized rollers 147 in a receptacle 104. The sensor 150 in the input port of the receptacle 104 is placed to respond to the initial insertion or retraction of the medical device by an operator. Other feed, clamp and eject mechanisms are also contemplated. The feed, clamp and eject mechanisms may be employed alone or in combination.

The proximal end portion 106 is received in a receptacle 134 of a contra connection device 104. The contra connection device 104 may be included on a workstation, computer, lab equipment, a driving device or sensing device or any other system or device. The contra connection device 104 makes electrical, mechanical and/or optical connections to equipment configured to interact with the device 102. As such, the contra connection device 104 includes active contacts that correspond with the active contacts of the device 102. For example, the optical fiber or light guide connection 110 corresponds with optical fiber or light guide connection 130 on the contra connection device 104; the electrical connections 114 (e.g., power and/or signal) correspond with electrical connections 136 on the contra connection device 104; the electrical pin connection 108 corresponds with an electrical slot or socket 128 on the contra connection device 104; lateral electrical connections 112 correspond with connections 132 on the contra connection device 104, etc. Other active or passive connections or features may be included, for example, mechanical connections may be made for gas or fluid flow or to form a lumen where materials or tools may be passed through the receptacle 104 into the proximal end portion 106. It should be understood that the active connections described and shown in FIG. 1 are illustrative and should not be construed as limiting.

The contra connection device 104 includes one or more ports in fluid communication with the receptacle 134. The ports include inlet ports 120 and 122 and outlet ports 124 and 126. It should be understood that the number and the types of ports may vary depending on the application and the type of flow desired. In one embodiment, the contra connection device 104 is configured to receive the proximal portion 106 of the device 102. The receptacle 134 may include a seal or seals 138 formed or mounted therein, e.g., an o-ring seal. The seal 138 is provided about an interior surface of the receptacle 134 to provide a fluid seal with the proximal end portion 106. The seal 138 is optional and can be omitted in some embodiments (e.g., where a seal is difficult to provide due to an outer texture of the device 102). Once the proximal end portion 106 is within the receptacle 134, the seal 138 prevents fluid from escaping along a longitudinal direction down the proximal end portion 106.

When the end portion 106 is inside the receptacle 134, at least one inlet 120 receives a jet 140 of cleaning fluid to wash, flush or sterilize the end portion 106. In one example, the jet 140 includes a liquid, such as distilled water, water, a non-conducting cleaning agent, alcohol, sterilizing liquid, etc. The jet 140 may be pressurized to enhance its cleaning ability. The fluid of the jet 140 will fill a remaining portion of the receptacle 134 not occupied by the device 102, and the fluid will be ejected from the outlet ports 124 and/126. Is should be noted that the jet 140 may be introduced in more than one inlet port 120, 122 or the jet 140 may be introduced through a single port 120 or 122.

In one embodiment, a second jet 142 may be introduced at a same time as the first jet 140 or the jets 140 and 142 may be introduced sequentially. For example, in one embodiment as described in detail with respect to FIGS. 2A-2C, the end portion 106 is moved so that its end lines up in the stream of the first jet 140. The first jet 140 employs a liquid cleansing process. Then, the end portion 106 is further advanced into the receptacle 134 to line up with a stream of the second jet 122. The second jet 142 may include a gas, such as, e.g., air or heated air. Other gases may also be employed. The second jet 142 may be employed to clear away any remaining debris and/or to dry off the proximal end portion 106 to prepare the end portion 106 for connection with the active connections of the contra connection device 104.

In one embodiment, the ports 120, 122, 124 and 126 may be selectively opened and closed. This may be through the use of valves on the contra connection device 104 or through valves on other equipment in communication with ports 102, 122, 124 and 126.

The contra connection device may include other features as well. For example, the receptacle 134 may include an indexing mechanism 143 configured to define the orientation of the device 102 with respect to the contra connection device 104, such that all connections are automatically aligned. The indexing mechanism 143 may include a flat, a torque stop, a keyway or other mechanical mechanism. The contra connection device 104 may be employed as a point of reference, e.g., for fiber optical shape sensing technology. Direction and position of the shape reconstruction may start from a position in the contra connection device 104 if the contra connection device 104 can be oriented in space in a reproducible way, for example, if the contra connection device 104 is mounted to a tracked flexible arm or to a fixation point on a patient table, etc. Other embodiments may include a flared or trumpet-shaped opening into the receptacle 134, which has a radius of curvature larger than or equal to a minimum bending radius of the device 102 (e.g., a guide wire). This can prevent kinking of the device 102. In still other embodiments, the contra connection device 104 may include non-metallic materials to be compatible with magnetic resonance imaging environments. Other embodiments may include automatic feed-in clamping, holding or ejection of the connection end of the medical device. This may be performed by motorized rollers 147, by a vacuum or by varying pressure or other mechanical connection devices inside the receptacle which may be triggered by the sensor 150 responding to the inserted end of the medical device.

FIGS. 2A-2C will now be described in accordance with one illustrative method. Referring to FIG. 2A, the proximal end portion 106 of the device 102 is inserted into the receptacle 134 and advanced to the first inlet port 120. An end of the end portion 106 is placed into an area corresponding with the first inlet 120. A first fluid jet 140 is activated to clean, flush or sterilize the end portion 106. The material of the first jet 140 is carried away through the outlets 124 and/or 126.

Referring to FIG. 2B, the proximal end portion 106 is further advanced into the receptacle 134 to the second inlet port 122. An end of the end portion 106 is placed into an area corresponding with the second inlet 122. A second fluid jet 142 is activated to clean, dry or sterilize the end portion 106. The material of the second jet 142 is purged through the outlets 124 and/or 126. In one example, the second jet 142 includes pressurized air employed to purge fluids and debris from the receptacle 134 in addition to drying surfaces in preparation for making active (e.g., electrical, optical, etc.) connections.

It should be understood that additional jets or treatment stations may be employed in the contra connection device 104. For example, it may be advantageous to provide an alcohol or other volatile liquid to clean off surfaces of optical fibers or light guides. In addition, jets may be applied from different directions at the same or different positions to enhance flushing, cleaning or drying the proximal end portion 106.

Referring to FIG. 2C, the device 102 is further advanced into the receptacle 134 to make a connection or connections between corresponding contacts for the active connections of the device 102 and the contra connection device 104. As described, the contacts may include connections that are optical, electrical, mechanical, etc. Once contact is made, the device 102 may be employed as intended for interventional procedures or the like. For example, in the cases of a guide wire, the guide wire may include optical fibers for shape sensing, electrical connections for powering a light or probe and signal leads for taking measurements or monitoring bodily functions. The guide wire (102) may be detached from the contra connection device 104 (to place objects on or remove objects from the guide wire) and reattached after using the purging mechanisms in accordance with the present principles to ensure a clean and functional active connection when attached and a sterilized proximal end of the medical device (the connection end) when detached.

Referring to FIG. 3, an illustrative system 200 is shown for cleaning a connector end of a device (102) in accordance with an exemplary embodiment. The device 102 and a contra connection device 104 may include the features and structures as described with reference to FIG. 1. The system 200 may include a workstation 202 configured to interact with the device 102 as needed to perform the functions the device 102 is designed for. For example, the device 102 may include optical fibers, and the work station 202 may include a program module or modules 240 for interpreting data feedback from the device 102 or providing control signals to the device 102, if needed. The data feedback may be received through an interface or cabling 222 (although wireless interfaces are contemplated as well). The cabling 222 may also provide control signals to the contra connection device 104 to control one or more features of the contra connection device 104 as will be described.

The workstation or console 202 is employed supervise and/or manage a procedure. Workstation 202 preferably includes one or more processors 212 and memory 214 for storing programs and applications. Memory 214 may store the program module or modules 240 and a control program 238. The control program 238 may be employed to generate control signals for controlling features of the contra connection device 104. These features may include for example, one or more valves 228, a pump 230, a compressor 232, a vacuum 234, sensors 242, a heating element(s) 246, etc.

While the workstation 202 and the features are optional for the operation of the cleaning functions of the contra connection device 104, the present embodiment provides for automated or semi-manually configurations for employing the present principles. In one embodiment, the contra connection device 104 includes sensors 242 corresponding to locations of inlet ports 120 and 124. When the end of the device 102 is present, the respective sensor 242 senses the event and provides feedback to the control program 238. The control program sends a control signal to turn on the pump 230 and open the valve 228 to permit a jet to clean the end portion of the device 102. In addition, the vacuum 234 may be turned on and its corresponding valves 228 may be opened to remove fluid and debris from the contra connection device 104. The vacuum 234 and valves 228 may be controlled by the control program as well, and may be employed with the feed, clamp and eject mechanism as described above. After a period, the jet and the sensor 242 corresponding to that jet are inactivated.

It should be understood that the sensors 242 may be employed with activating a feed, clamp and eject mechanism to receive and secure the proximal end of the medical device after an initial insertion or retraction of the medical device. The feed, clamp and eject mechanism may move and stop the medical device as needed to be treated by each stream in the receptacle 104. In one embodiment, the control program 238 may be employed to manage movement of the medical device and all cleaning activities.

As the device 102 is further inserted, the respective sensor 242 for a next port 122 senses the event and provides feedback to the control program 238. The control program 238 sends a control signal to turn on a compressor 232 (or heater or other device) and open a valve 228, e.g., using an actuator or the like, to permit a jet to clean/dry the end portion of the device 102. In addition, the vacuum 234 may be turned on and its corresponding valves 228 may be opened to remove air, fluid and/or debris from the contra connection device 104. The vacuum 234 and valves 228 may be controlled by the control program as well. After a period, the jet and the sensor 242 corresponding to that jet are inactivated. It should be understood that the compressor 232 may be replaced by another pump, a heater, an air tank, etc. It should be understood that the use of the vacuum 234 is optional, and that other configurations are contemplated that have various combinations of features, numbers of jets, types of jets (gas, liquid, etc.), numbers and types of valves, etc. It should also be understood that different levels of automation may be provided, for example, a switch 244, e.g., a foot switch, may be provided to enable the cleaning jet or jets for cleaning the device 102.

In one embodiment, the proximal end of the device 102 may have a high stiffness and can be inserted into a receiving end of the contra connection device 104, in an oriented manner. By insertion (using the sensor 242 or by other means), the fluid jet is activated to clean the proximal end of the device 102 and flush out waste. The first fluid jet may include, for example, water or a non-conductive cleaning agent. The fluid is ejected during the flushing of the proximal end of the device 102. A drying step may include using a same inlet port 120 with a second jet of air, heated air, filtered compressed air, other gas, etc. The drying step may also include employing a heating element 246 to evaporate remaining liquids or employing a liquid absorbing material. In one embodiment, a sterilization step may be performed concurrently or in addition to the flush and dry steps. The sterilization step may include using the heating element 246 or the application of a sterilizing fluid or hot gas at the insertion point.

Software modules in memory 214 run on the workstation 202. The control program 238 may be configured or customized using a display 224 and a user interface 226, which prompts and guides the user in interacting with the control program 238. The interface 226 may include a keyboard, mouse, a joystick, a haptic device, or any other peripheral or control to permit user feedback from and interaction with the workstation 202. For example, a sensor in the input port of the receptacle is placed to respond to the initial insertion or retraction of the medical device by an operator. This will allow an automatic method to feed, clamp and eject the proximal end of the medical device by using motorized rollers or gas pressure in the receptacle.

Referring to FIG. 4, a method for providing a connector for connection is illustratively shown. It should be understood that while the FIG. 4 is a flow diagram showing a method for inserting a connector, the method is applicable for ejecting, removing or unplugging the connector from a receptacle as well. For example, as the connector is withdrawn a cleansing or sterilizing process may be employed in reverse order switching and/or the jet types, etc. in accordance with illustrative embodiments.

In block 302, a medical device or other device may be employed in a procedure or other process. In one embodiment, the medical device may include a catheter or guide wire that is inserted within a patient. The guide wire may be detached from a receptacle to thread devices down the guide wire that are either inserted into or removed from the patient. This may result in bodily fluids or tissues including blood covering a proximal end portion of the guide wire or catheter. In block 304, a connecting end portion of a connection device, such as the guide wire or catheter, is inserted in a receptacle. An automatic insertion and hold mechanism may be employed. In block 305, the connecting end portion may be indexed to the receptacle to align active connections of the connecting end portion and active connections of the receptacle. This may also be employed to provide a reference position, e.g., for providing an origin for fiber optic sensing, etc.

In block 306, the connecting end portion is advanced into the receptacle to a first position corresponding with a first inlet port. In one embodiment, a feed, clamp and eject mechanism may be activated to receive and secure the proximal end of the medical device after an initial insertion or retraction of the medical device. This device may be employed to partially advance or retract the device in accordance with a number of ports/streams to be employed.

In block 308, a first fluid jet is streamed into the first inlet port to douse the connecting end portion. In block 309, the connecting end portion may be sensed by a sensor at the first position to enable the flow of the first fluid jet into the first inlet port. Additional sensors may be employed at other inlet positions.

In block 310, the receptacle is cleared to improve contact between active connections of the connecting end portion and active connections of the receptacle. The active connection may include electrical, mechanical and/or optical connections. Clearing the receptacle may include, in block 312, advancing the connecting end portion into the receptacle to a second position corresponding with a second inlet port, and in block 314, streaming a second fluid jet into the second inlet port to douse the connecting end portion. The first fluid jet may include a liquid (e.g., water, alcohol, etc.) and the second fluid jet may include a gas (e.g., air, nitrogen, etc.). In block 315, the connecting end portion may be sensed by a sensor at the second position to enable the flow of the second fluid jet into the first inlet port. Additional sensors may be employed at other inlet positions.

In block 316, the first fluid jet may include a liquid, and the receptacle may be cleared by drying the liquid with a gas purge. In block 318, the first fluid jet may include a liquid, and the receptacle may be dried by heating the receptacle.

In block 320, the streaming and/or the clearing may include sterilizing the connecting end portion. In block 322, a full connection is made between corresponding active connection points of the receptacle and the connecting end portion. The active connection points may include corresponding electrical, optical and/or mechanical connections.

With respect to the electrical connections in particular, it should be noted that non-corrosive fluids should be used. The proper working of all connections, either electrical, optical could be tested automatically upon reconnection, for example, by measuring system impedances or optical reflections. The connector may be withdrawn from the receptacle, its presence sensed at or near the inlet ports and cleaned using the jets as described for the insertion steps. Any combination of steps may be employed for the cleaning or sterilization processes.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for a flushed medical connector with optical and electrical connections (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A medical connector receptacle, comprising:
a receptacle configured to receive a proximal end portion of a medical device;
at least one inlet port in fluid communication with the receptacle and configured to receive a
fluid for cleaning the proximal end portion;
at least one outlet port in fluid communication with the receptacle and configured to eject the fluid and debris from the receptacle; and
at least one active connection point configured to mate with the proximal end portion of the medical device such that the fluid removes the debris to permit an improved connection.

2. The medical connector receptacle as recited in claim 1, wherein the receptacle includes a seal to seal to the proximal end portion of the medical device.

3. The medical connector receptacle as recited in claim 1, wherein the receptacle includes an index mechanism to provide a repeatable connection orientation with the proximal end portion of the medical device.

4. The medical connector receptacle as recited in claim 1, wherein the least one inlet port includes two inlet ports and each inlet port provides a stream of a different material, wherein the two inlet ports provide a liquid stream and a gas stream, and wherein the fluid includes one or more of air, water and a sterilizing fluid.

5. The medical connector receptacle as recited in claim 1, wherein the active connection points include a combination of electrical and optical connection points.

6. The medical connector receptacle as recited in claim 1, further comprising:
a feed, clamp and eject mechanism to receive and secure the proximal end of the medical device; and
a sensor mounted within the receptacle to respond to an initial insertion or retraction of the medical device.

7. The medical connector receptacle as recited in claim 1, wherein the feed, clamp and eject mechanism includes one of a gas pressure regulation mechanism and motorized rollers in the receptacle.

8. A system for cleaning connections, comprising:
a medical connector receptacle according to claim 1;
at least one valve coupled to the at least one inlet port, the at least one valve being controlled to enable or disable a fluid jet to clean the proximal end portion; and
a sensor disposed within the receptacle to enable the at least one valve when the proximal end portion is sensed in the receptacle.

9. The system as recited in claim 8, further comprising a vacuum coupled to the at least one outlet port to draw the fluid from the receptacle.

10. The system as recited in claim 8, wherein the medical connector receptacle further includes an index mechanism to provide a repeatable connection orientation with the proximal end portion of the medical device.

11. The system as recited in claim 8, wherein the least one inlet port includes two inlet ports and each inlet port includes an associated valve that provides a stream of a different material, wherein the two inlet ports provide a liquid stream and a gas stream, and wherein the fluid includes one or more of air, water and a sterilizing fluid.

12. The system as recited in claim 8, wherein the active connection points include a combination of electrical and optical connection points.

13. The system as recited in claim 8, further comprising a controller configured to receive feedback from the receptacle on a position of the proximal end portion of the medical device such that one or more jets are enabled based upon the position of the proximal end portion, wherein the one or more jets are enabled by opening the at least one valve to release a pressurized fluid, and wherein the pressurized fluid originates from one or more of a pump, a compressor and a tank.

14. The system as recited in claim 8, wherein the medical connector further includes:
a feed, clamp and eject mechanism to receive and secure the proximal end of the medical device responsive to an initial insertion or retraction of the medical device, wherein the feed, clamp and eject mechanism includes one of a gas pressure regulation mechanism and motorized rollers in the receptacle.

15. (canceled)

16. The system as recited in claim 8, wherein the medical connector receptacle further includes:
a sensor mounted within the receptacle to respond to an initial insertion or retraction of the medical device.
